(19) United States
(12) Patent Application Publication
(10) Pub. No.: US 2007/0169285 A1
(43) Pub. Date: Jul. 26, 2007

(54) Method and kit for coloring hair

(76) Inventors: Saroja Narasimhan, Matawan, NJ (US); Lou Ann Christine Vena, Scotch Plains, NJ (US); Shailendra Kumar Singh, Edison, NJ (US); Dolores Barbara Cross, South Amboy, NJ (US)

Correspondence Address: Julie Blackburn;Revlon Consumer Products Corporation, 237 Park Avenue, New York, NY, 10017, US

(21) Appl. No.: 11/339,278

(22) Filed: Jan. 25, 2006

(51) Int. Cl. A61K 8/00

(52) U.S. Cl. 8/405

(57) ABSTRACT

A kit for coloring hair containing at least one leave on pretreatment composition for application to hair prior to coloring, and a method for coloring hair and improving hair condition and evenness of color deposition in hair that is permanently colored or lightened.

### TECHNICAL FIELD

The invention is in the field of processes and kits for coloring hair.

### BACKGROUND OF THE INVENTION

There are three general types of hair color: temporary, semi-permanent, or oxidative. Temporary hair color typically refers to rinses that are applied to the hair and last until they are shampooed out. Examples of temporary hair color rinses are those used by individuals who want a specific color effect for an abbreviated period of time (such as green hair for St. Patrick's Day).

Semi-permanent hair color refers to hair color achieved with the so-called direct dyes. Direct dyes may be applied directly to the hair to provide color. They do not require mixing with an oxidizing agent to activate. Semi-permanent dyes generally last anywhere from about six to twenty four shampoos and are believed to coat the hair fiber with color rather than permeating into the hair shaft.

Oxidative dyes, also referred to as permanent dyes, provide permanent coloration to the hair. The composition containing the oxidative dyes is combined with an activating composition, generally referred to as a developer. The mixture is applied to the hair and after the appropriate period of time will impart permanent color to the hair. While oxidative color is permanent, hair that is oxidatively colored must be touched up every six to eight weeks to color new hair growth or strands that have faded.

While oxidative color provides the most permanent result, the process can also cause the hair to become dry or damaged when used too often or by individuals that have overly sensitive hair. When hair is damaged, the hair color process does not yield optimal results. In particular, damaged hair exhibits variations in cuticle thickness and porosity. Often the ends of the hair are most damaged and porous. Thus, when an oxidative dye is applied to such hair, more dye will be absorbed into the more porous and damaged regions of the hair and less into the other portions of the hair. The resulting hair dye procedure provides hair color that is not uniform. In some cases where there is a great disparity in porosity and texture between the hair strands, the color can be noticeably different in different sections of the hair. Further, the increased absorption of the hair dye mixture by the more damaged sections of the hair may promote further hair damage. Yet, those who oxidatively color their hair are not inclined to discontinue their regular color process just because their hair becomes dry or brittle.

Accordingly, there is a need for a pre-treatment composition that can be applied to hair prior to hair dyeing or bleaching, that will protect and condition the hair and promote uniformity and evenness of hair coloration, yet not interfere with the coloring or bleaching process. Ideally, the oily ingredient must be one that is preferentially absorbed into the hair shaft. It is well known to pre-treat hair with various conditioning compositions. However, it is conventional wisdom that such compositions interfere with deposit of color in hair coloring procedures.

It is an object of the invention to provide a composition for use in pre-treating hair prior to coloring or bleaching, that will provide improved hair conditioning, more uniform color deposit, and which does not interfere with color deposit.

It is a further object of the invention to provide a process for pre-treating hair prior to coloring or bleaching to promote more even color deposition in the colored or bleached hair.

It is a further object of the invention to provide an oxidative hair color kit containing an oxidative hair dye composition, a developer composition, and a pre-treatment composition.

It is a further object of the invention to provide a hair bleach kit containing a persulfate bleach composition, a developer composition, and a pre-treatment composition.

It is a further object of the invention to provide a hair color kit containing a semi-permanent dye composition and a pre-treatment composition.

It is a further object of the invention to provide a hair highlighting kit containing a developer, a persulfate bleach composition, a pre-treatment composition, and an applicator for the highlights or a cap and hook.

It is a further object of the invention to provide a hair lowlighting kit containing a lowlight dye composition, a developer, a pre-treatment composition, and an applicator for applying the lowlight dye mixture to the hair.

### SUMMARY OF THE INVENTION

The invention is directed to an oxidative hair color kit comprising at least: (a) a receptacle containing an oxidative hair color composition, (b) a receptacle containing a developer composition, wherein (a) and (b) are combined to form a hair color mixture; and (c) a receptacle containing at least one leave on pretreatment composition for application to the hair prior to coloring the hair with the mixture of (a) and (b).

The invention is further directed to a semi-permanent hair color kit comprising (a) a receptacle containing a semi-permanent dye composition, and (b) a receptacle containing a leave on pretreatment composition for application to the hair prior to coloring the hair with the semi-permanent dye composition.

The invention is further directed to a hair bleach kit comprising (a) a receptacle containing a developer, (b) a receptacle containing a persulfate bleach composition, and (c) a receptacle containing a leave on pretreatment composition for application to the hair prior to bleaching the hair with the hair bleach mixture formed by combining (a) and (b).

The invention is further directed to a hair highlighting kit comprising (a) a receptacle containing a developer composition, (b) a receptacle containing a persulfate bleach composition, (c) at least one application device, and (d) a receptacle containing a leave on pretreatment composition for application to the hair prior to highlighting the hair with the mixture of (a) and (b).

The invention is further directed to a lowlighting kit comprising (a) a receptacle containing a developer composition, (b) a receptacle containing a lowlight dye composition, (c) an applicator; and (d) a receptacle containing a leave on pretreatment composition for application to the hair prior to applying lowlights to the hair with the applicator and the mixture of (a) and (b).

The invention is also directed to a method for coloring hair comprising:
(a) applying a leave on pretreatment composition to the hair to be colored,
(b) coloring the hair with a hair coloring composition applied over the pretreatment composition.

The invention is further directed to a method for improving hair conditioning and/or evenness of color deposition in hair that is colored comprising applying a leave on pretreatment composition containing a low molecular weight diffusible oil to the hair prior to coloring the hair.

### BRIEF DESCRIPTION OF THE DRAWINGS

FIG. 1: depicts a kit for oxidatively coloring hair which contains developer, the pretreatment composition, the oxidative dye composition, an after treatment hair conditioner, and a container for storing the items in the kit.

FIG. 2: depicts a kit for coloring hair with semi-permanent hair color containing a semi-permanent dye composition, a pretreatment composition, an after treatment conditioner, and a container for storing the items in the kit.

FIG. 3: depicts a kit for bleaching the hair containing a developer, a persulfate composition, a pretreatment composition, an after treatment composition, and a container for storing the items in the kit.

FIG. 4: depicts a kit for highlighting the hair containing a developer, a persulfate composition, a pretreatment composition, an after treatment composition, an applicator used to apply the highlight mixture to the hair, and a container for storing the items in the kit.

FIG. 5: depicts a kit for lowlighting hair containing a developer, a lowlight dye composition, a pretreatment composition, an after treatment conditioner, an applicator for applying the lowlight dye mixture to the hair, and a container for storing the items in the kit.

### DETAILED DESCRIPTION

The invention comprises a hair color kit containing the pretreatment composition, a method for coloring hair, and a method for improving the condition and even coloration of dyed hair.

I. The Hair Color Kit

A variety of hair color kits are encompassed within the scope of the invention. Such kits contain the items needed to impart color to the hair and include oxidative hair color kits, semi-permanent hair color kits, hair bleaching kits, hair highlighting kits, hair lowlighting kits, and the like.

A. Oxidative Hair Color Kit

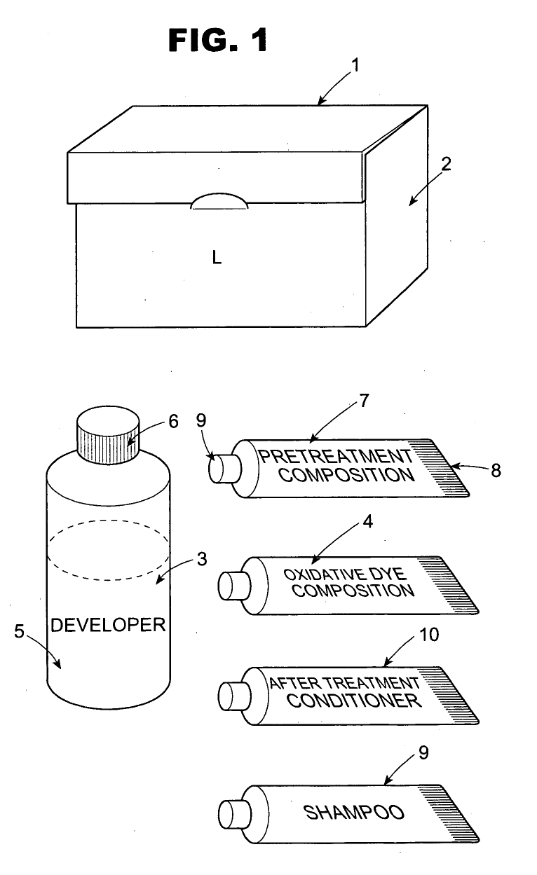

One embodiment of the invention is directed to an oxidative hair color kit 1 as depicted in FIG. 1. The kit comprises a container 2 or carton for storing the items in the kit. The container 2 may be made of plastic, cardboard or paper. The container 2 may be printed with graphics, text, photographs, or any other indicia that the manufacturer may wish to use to characterize the product.

The kit 1 contains a developer composition 3. The developer composition 3 is generally an aqueous based oxidizing agent composition that, when combined with the oxidative dye composition 4, also found in the kit 1 forms a mixture capable of imparting permanent color to hair. The oxidizing agent most often used in such developer compositions is hydrogen peroxide. The developer composition 3 is preferably contained in a bottle 5, preferably plastic, that is closed with a closure 6, most preferably a screw on cap.

The kit 1 also contains the leave on pretreatment composition 7, which is contained in a tube 8, bottle, packette, or similar type of container. The tube 8 is preferably closed with a closure 9.

Kit 1 may optionally contain an after treatment shampoo 9 that is applied to the hair to cleanse it after conclusion of the oxidative dye process. While shampoo 9 may be found in a variety of different types of containers, the preferred container is a tube.

Kit 1 may also optionally contain an after treatment hair conditioner 10 that is used to condition the hair after it has been oxidatively dyed and washed with shampoo 9. While the after treatment conditioner may be found in a variety of different receptacles, preferred is where the conditioner is found in a tube.

The oxidative hair color kit 1 may contain other items including but not limited to gloves, directions, package inserts, discount coupons, diagrams, booklets, free samples of other manufacturer products, and so on.

The kit 1 may be imprinted with photographs of models having the hair color to be delivered when using the kit, the ingredient listing for the products found within the kit 1, graphics and designs that appeal to shoppers, and so on.

B. Semi-Permanent Hair Color Kit

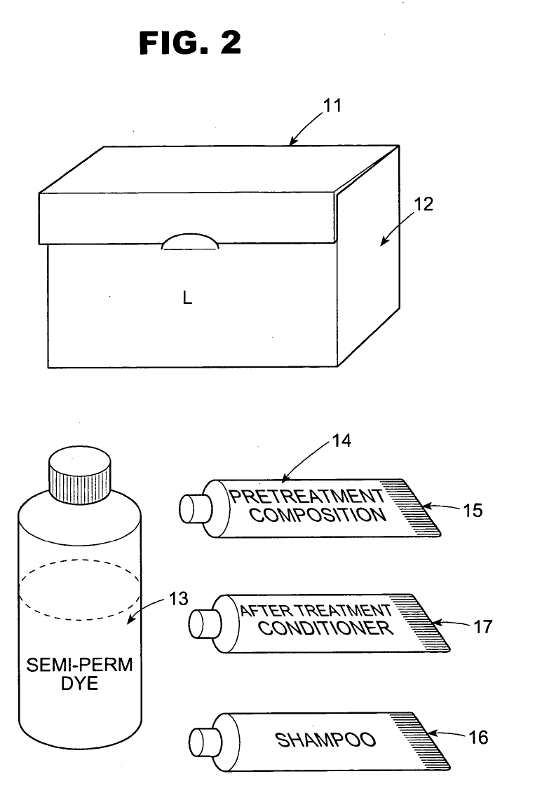

The invention may also be directed to a kit for coloring the hair with semi-permanent hair color as depicted in FIG. 2. Semi-permanent hair color differs from oxidative color in that the dyes are directly applied to the hair and will impart color without the necessity of combining the dyes with an oxidizing agent. Semi-permanent color generally washes out in six to twenty four shampoos depending on strength and intensity.

The semi-permanent hair color kit 11 is depicted in FIG. 2. The kit 11 comprises a container 12 that is suitable for containing the various items found in the kit 11.

Kit 11 contains a semi-permanent dye composition 13 that generally comprises semi-permanent or direct dyes in an aqueous solution or suspension. The semi-permanent dye composition 13 is capable of imparting color to the hair directly without combining with oxidizing agents.

The kit 11 also contains a leave on pretreatment composition 14 that is applied to hair prior to treating the hair with the semi-permanent dye composition 13. The pretreatment composition may be contained in a variety of receptacles, a tube 15 being one. Bottles, packettes, and the like are also suitable.

If desired, the kit 11 may optionally contain a shampoo composition 16 for use in shampooing the hair after completion of the color process with the semi-permanent dye.

Kit 11 may also optionally contain an after treatment conditioner composition 17, to be used to condition the hair treatment with the semi-permanent dye, or if the hair is shampooed, the shampoo.

Container 12 may also contain a variety of other items such as gloves, package inserts, discount coupons, promotional materials, and so on.

C. Hair Bleach Kit

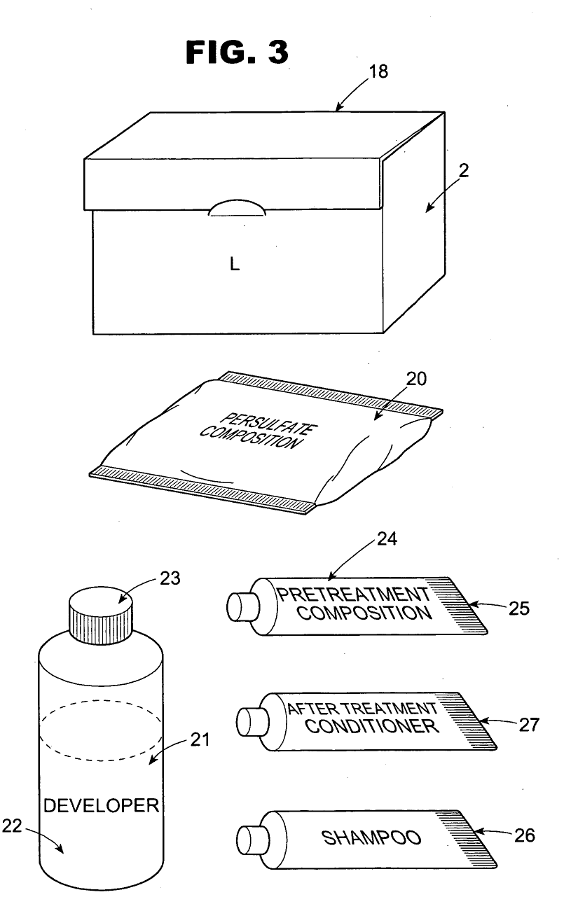

One embodiment of the invention is directed to a hair bleach kit 18 as depicted in FIG. 3. The kit 18 comprises a container 19 or carton that is suitable for storing the other items in the kit 18.

Kit 18 contains a persulfate composition 20 that is generally comprised of particulate persulfate salts that are operative to bleach hair when combined with a developer 21. Developer 21 is preferably contained in a plastic container such as a bottle 22 having a screw cap 23.

Kit 18 also contains pretreatment composition 24. While the receptacle shown for the pretreatment composition 24 is a tube 25, a variety of other receptacles are suitable in including jars, bottles, packettes, and so on.

Optionally, kit 18 may contain a shampoo composition 26 for use in shampooing the hair after it has been treated with the bleach composition.

Kit 18 may also optionally contain an after treatment hair conditioner composition 27, for use in treating the hair after it has been bleached, or bleached and shampooed.

As with the other kits of the invention, the kit 18 may contain other items such as discount coupons, promotional materials, gloves, directions for using the products in the kit and so on.

D. Hair Highlighting Kit

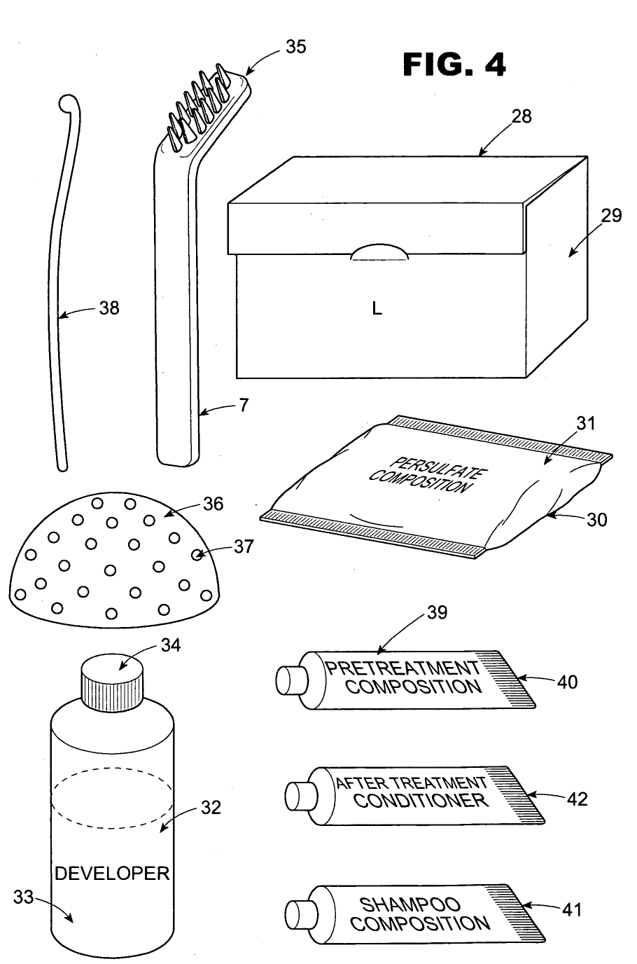

One embodiment of the invention comprises a hair highlighting kit 28 as depicted in FIG. 4. The kit 28 comprises a container 29 or carton suitable for containing the various items found in the kit 28. As with the other kits, the container 29 may be made of plastic, cardboard, paper or any other suitable material. Preferably it is made from cardboard or paper.

Highlighting kit 28 contains a persulfate composition 30 which may be contained in a variety of containers, but the preferred receptacle is a packette 31.

Highlighting kit 28 further contains a developer 32 for combining with the persulfate composition 30 to form a hair highlighting mixture. Developer is preferably contained in a plastic bottle 33 with a closure that is a screw cap 34.

Highlighting kit 28 contains an applicator 35 for use in applying the hair highlighting composition to sections of the hair. Alternatively, a cap 36 with holes 37 may be included in the kit 28. Cap 36 is placed on the head and strands of hair are pulled through holes 37 using a hook 38. In the latter case, the highlighting procedure is referred to as the “cap and hook” method.

Kit 28 further contains the pretreatment composition 39. Pretreatment composition 39 may be stored in a tube 40 as depicted, or other receptacles such as packettes, jars, bottles, and the like.

Kit 28 may optionally contain a shampoo composition 41 that is used to cleanse the hair after the highlighting process has been completed.

Kit 28 may also optionally contain an after treatment conditioner composition 42. The after treatment conditioner may be contained in a variety of other receptacles other than the tube that is depicted.

The container 29 is large enough to hold the essential items in the kit 28. In addition, other items may be included in the kit such as package inserts, discount coupons, promotional materials, instructions, protective gloves and so on.

E. Hair Lowlighting Kit

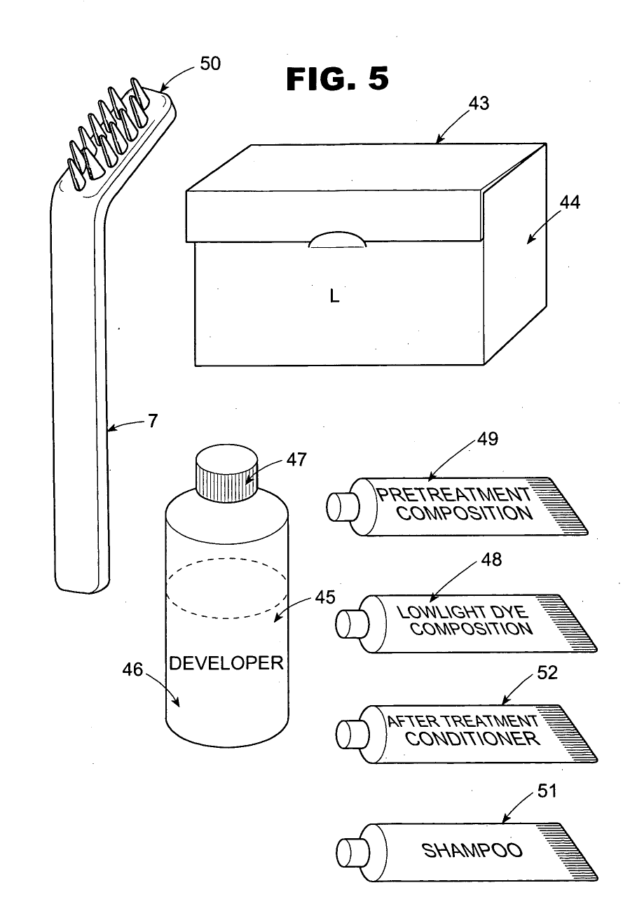

One embodiment of the invention is a hair lowlighting kit 43 as depicted in FIG. 5. The kit 43 comprises a container 44 or carton that may be made from plastic, cardboard, paper, or any other suitable material.

Kit 43 contains a developer composition 45, which is preferably contained in a plastic bottle 46 with a screw cap 47.

Kit 43 also contains a lowlight dye composition 48, which is generally an oxidative hair color composition. The lowlight dye composition 48 may be contained in a variety of other types of receptacles other than the tube that is depicted.

Kit 43 further contains a leave on pretreatment composition 49. Again, the leave on pretreatment composition may be found in a variety of other receptacles other than the tube that is depicted, such as jars, bottles, packettes, and the like.

Kit 43 also contains an applicator 50 used for applying the lowlight dye mixture obtained when combining the lowlight dye composition 48 and the developer composition 45. The applicator is used to apply the lowlight dye mixture to select strands of hair to achieve the desired effect.

Kit 43 may optionally contain a shampoo composition 51 for use in cleansing the hair after the lowlighting process. Shampoo composition 51 may be contained in a variety of other types of receptacles other than the tube that is depicted.

Kit 43 may also optionally contain an after treatment hair conditioner 52 for use in conditioning the hair after conclusion of the lowlighting process or the shampoo process if the shampoo is used.

With respect to all of the kits mentioned herein, they may contain package inserts such as discount coupons, promotional literature, instructions for using the components of the kits, applicators, free items, protective gloves, and so on.

The kits may be imprinted with graphics, photographs, drawings, ingredient listings, and any number of designs, indicia, or other information.

In all cases the items in the kit are used in the manner described in the instructions to provide the user with the desired hair color. In all cases, the pretreatment composition found in the kit is applied to the hair first, prior to coloring the hair.

II. The Method

The invention is further directed to a method for coloring hair and a method for improving the condition and evenness of color deposition on hair using the pretreatment composition of the invention.

A. The Method for Coloring Hair

The invention comprises a method for coloring hair comprising, first, (a) applying a leave on pretreatment composition to the hair to be colored, then (b) coloring the hair with the hair color composition.

1. The Pretreatment Composition

The pretreatment composition comprises at least one low molecular weight diffusible oil that is operative to be preferentially absorbed into the hair depending on hair damage and porosity. For example, where hair is more porous and damaged (for example, at the ends), the oil will be more effectively absorbed. In areas of the hair shaft that are less porous or damaged, the oil will form a very light film with less absorption into the hair shaft than found in the porous areas of the hair.

The term “low molecular weight” when used herein, generally means that the diffusible oil will have a molecular weight ranging from about 100 to 5000 Daltons, preferably from about 200 to 3000 Daltons, more preferably from about 500 to 1500 Daltons.

The term “diffusible” when used herein, generally means that the oil will be capable of being absorbed, or diffusing, into the areas of the hair shaft that are most damaged and porous, yet remaining on the surface of the hair strand sections that have a healthy cuticle.

The term “oil” means a substance that is a pourable liquid at a temperature ranging from about 25 to 35° C.

Suitable oils include coconut oil, mineral oil, almond oil, apricot kernel oil, avocado oil, babassu oil, evening primrose oil, camelina sativa seed oil, grape seed oil, macadamia ternifolia seed oil, corn oil, meadowfoam seed oil, mink oil, olive oil, palm kernel oil, safflower oil, sesame oil, soybean oil, sunflower oil, wheat germ oil, camellia reticulata seed oil. Also included as suitable oils are esters that are classified by the CTFA Cosmetic Ingredient Dictionary/Handbook, Eighth Edition, 2000 as “skin conditioning agents-emollients”. The CTFA Dictionary/Handbook defines such ingredients as those that help to maintain the soft, smooth, and pliable appearance of skin. Emollients function by their ability to remain on the skin surface or in the stratum corneum to act as lubricants, to reduce flaking, and to improve the skin's appearance.

In the case where such oils are esters, they are preferably esters of C₁₋₁₀ carboxylic acids and C₁₋₃₀ mono or dihydric alcohols, more preferably monoesters or diesters. Even more preferred is where such esters have a molecular mass ranging from about 200 to 600 grams/mol. Such esters include esters of isopropyl alcohol such as isopropyl myristate, oleate, palmitate, stearate, isostearate, and so on. Also suitable are esters of isononanoic acid such as isononyl isnonanoate, isopropyl isononanoate; esters of malic acid such as diisostearyl malate, diisopropyl adipate, isopropyl adipate, isostearyl malate, and so on.

The pretreatment composition may contain one or more oils. Preferably it comprises coconut oil alone or in combination with mineral oil. If the latter, the oily composition comprises from about 40 to 60 parts of coconut oil and from about 40 to 60 parts of mineral oil.

2. The Hair Color

After the leave on pretreatment composition is applied the hair, the hair may be colored with oxidative dye, semi-permanent dyes, hair bleaches, hair highlighting composition, hair lowlighting compositions, and the like. The color is applied to the hair on top of the leave on pretreatment composition and the hair color method is conducted according to the normal process.

(a). Oxidative Dye Mixture

One type of hair color that may be used in the method of the invention is an oxidative dye mixture that is prepared by combining an oxidative dye composition and a developer composition to form a mixture that is capable of imparting permanent color to hair.

(i). Oxidative Dye Composition

The oxidative dye composition generally comprises an aqueous base containing primary intermediates and couplers.

(aa). Water

The Oxidative dye composition contains water ranging from about 5-95%, preferably from about 10-85%, more preferably from about 15-80% by weight of the total composition.

(bb). Primary Intermediates and Couplers

The oxidative dyes found in the oxidative dye composition are generally in the form of primary intermediates and couplers. Suggested ranges of primary intermediates are about 0.0001-15%, preferably about 0.0005-12%, more preferably about 0.001-10% by weight of the total composition. Such primary intermediates are well known for use in hair color, and include ortho or para substituted aminophenols or phenylenediamines, such as para-phenylenediamines of the formula: 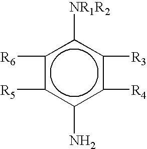
wherein R₁ and R₂ are each independently hydrogen, C₁₋₆ alkyl or C₁₋₆ alkyl substituted with one or more hydroxy, methoxy, methylsulphonylamino, aminocarbonyl, furfturyl, unsubstituted phenyl, or amino substituted phenyl groups; R₃, R₄, R₅, and R₆ are each independently hydrogen, C₁₋₆ alkyl, C₁₋₆ alkoxy, halogen, or C₁₋₆ alkyl substituted with one or more hydroxy or amino groups.

Specific examples of suitable primary intermediates include para-phenylenediamine, 2-methyl-1,4-diaminobenzene, 2,6-dimethyl-1,4-diaminobenzene, 2,5-dimethyl-1,4-diaminobenzene, 2,3-dimethyl-1,4-diaminobenzene, 2-chloro-1,4-diaminobenzene, 2-methoxy-1,4-diaminobenzene, 1-phenylamino-4-aminobenzene, 1-dimethylamino-4-aminobenzene, 1-diethylamino-4-aminobenzene, 1-bis(beta-hydroxyethyl)amino-4-aminobenzene, 1-methoxyethylamino-4-aminobenzene, 2-hydroxymethyl-11,4-diaminobenzene, 2-hydroxyethyl-1,4-diaminobenzene, 2-isopropyl-1,4-diaminobenzene, 1-hydroxypropylamino-4-aminobenzene-, 2,6-dimethyl-3-methoxy-1,4-diaminobenzene, 1-amino-4-hydroxybenzene, and derivatives thereof, and acid or basic salts thereof.

Preferred primary intermediates are p-phenylenediamine, p-aminophenol, o-aminophenol, N,N-bis(2-hydroxyethyl)-p-phenylenediamine, 2,5-diaminotoluene, their salts and mixtures thereof.

(cc) Color Couplers

The dye composition may comprise one or more color couplers in ranges of about 0.0001-10%, more preferably 0.0005-8%, most preferably 0.001-7% by weight of the total composition. Suitable color couplers include, for example, those having the general formula: 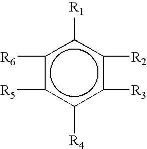
wherein R₁ is an unsubstituted hydroxy or amino, or hydroxy or amino substituted with one or more C₁₋₆ hydroxyalkyl groups, R.sub.3 and R.sub.5 are each independently hydrogen, hydroxy, amino, or amino substituted with C₁₋₆ alkyl, C₁₋₆ alkoxy, or C1-6 hydroxyalkyl group; and R₂, R₃R₄, R₅ and R₆ are each independently hydrogen, C₁₋₆ alkoxy, C₁₋₆ hydroxyalkyl, or C₁₋₆ alkyl, or R₃ and R₄ together may form a methylenedioxy or ethylenedioxy group.

Examples of such compounds include meta-derivatives such as phenols, catechol, meta-aminophenols, meta-phenylenediamines, and the like, which may be unsubstituted, or substituted on the amino group or benzene ring with alkyl, hydroxyalkyl, alkylamino groups, and the like. Suitable couplers include m-aminophenol, 2,4-diaminotoluene, 4-amino, 2-hydroxytoluene, phenyl methylpyrazolone, 3,4-methylenedioxyphenol, 3,4-methylenedioxy-1-[(beta-hydroxyethyl)amino]-benzene, 1-methoxy-2-amino-4-[(beta-hydroxyethyl)amino]benzene, 1-hydroxy-3-(dimethylamino)benzene, 6-methyl-1-hydroxy-3[(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-methoxy-1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene-, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl-1,3-diaminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, 2,4-diamionphenoxyethanol, and mixtures thereof.

Preferred couplers include resorcinol, 1-naphthol, 2-methylresorcinol, m-aminophenol, 2,4-diaminophenoxyethanol, phenyl methylpyrazolone, their salts, or mixtures.

Preferably the oxidative dye composition comprises one or more primary intermediates and one or more color couplers in the amounts set forth.

The oxidative dye composition may contain other ingredients including but not limited to humectants, surfactants, hair conditioning ingredients, and so on. Examples of oxidative dye compositions are set forth in U.S. Pat. No. 5,843,193; and U.S. Patent Publication Nos. 2005/0125913; 2005/0097684; 2005/0028833; 2004/0244126; 2004/0154108; 2004/0045101; and 2004/0016064; all of which are hereby incorporated by reference in their entirety.

(ii). The Developer

The developer composition that is found in the kits is generally an aqueous oxidizing agent composition comprising from about 1-99%, preferably from about 5-95%, more preferably from about 10-95% by weight of the total composition of an oxidizing agent, which is preferably hydrogen peroxide. The developer composition may additionally contain other ingredients such as oils, surfactants, humectants, preservatives, and so on as set forth in the patents and patent publications mentioned with respect to the oxidative dye composition.

Other ingredients and developer compositions suitable for use in the kits are as described in the U.S. Patents and Patent Publications referenced herein.

(b). Semi-Permanent Dye

The kit and method may use a semi-permanent dye to color the hair after application of the leave on pretreatment composition. Generally, the semi-permanent dye composition is aqueous based, comprising from about 1-99%, preferably from about 5-95%, more preferably from about 10-90% by weight of the total composition of water, and one or more semi-permanent dyes including those further described herein. The semi-permanent dye may be water soluble or water dispersible, with such dyes being operable to color hair when directly contacted with the hair and without the necessity of pre-combining with a developer.

A variety of dyes are suitable including direct dyes, disperse dyes, acid dyes, basic, dyes, direct, dyes, and so on. Suitable amounts of dye preferably range from about 0.0001-20%, preferably about 0.005-15%, more preferably about 0.010-10% by weight of the total composition. Preferred are the compounds that fall into the general category of semi-permanent dyes. Examples of such dyes are set forth below:

(i). Basic Dyes

Suitable basic, or cationic dyes include blues, browns, greens, oranges, reds, and yellows. Suitable blues include Basic Blue 3, 6, 7, 9, 26, 41, 47, and 99. Suitable browns include Basic Browns 4, 16, and 17. Suitable greens include Basic Green 1 and 4. Suitable oranges include Basic Orange 1 and 2. Suitable Reds include Basic Red 1, 2, 22, 46, 76, and 118. Suitable violets include Basic Violet 1, 3, 4, 10, 11:1, 14, and 16. Suitable yellows include Basic Yellow 11, 28, and 57.

Suitable basic dyes for use in the claimed compositions are set forth in the CTFA Cosmetic Ingredient Handbook, Eighth Edition, pages 117-124, which are hereby incorporated by reference in their entirety.

(ii). HC Dyes

Also suitable for use in the compositions are various HC dyes such as blue, brown, green, orange, red, violet, and yellow. Suitable blues include HC Blue 2, 4, 5, 6, 7, 8, 9, 10, 11, 12, 13, and 14. Suitable browns include HC Brown I and 2. Suitable greens include HC Green 1. Suitable oranges include HC Orange 1, 2, 3, and 5. Suitable reds include HC Red 1, 3, 7, 8, 9, 10, 11, 13, and 14. Suitable violets include HC Violet 1 and 2. Suitable yellows include HC Yellow 2, 4, 5, 6, 7, 8, 9, 10, 11, 13, 14, and 15. Such HC dyes are set forth on pages 615-623 of the CTFA Cosmetic Ingredient Handbook, Eighth Edition, 2000, which is hereby incorporated by reference in its entirety.

(iii). Acid Dyes

Also suitable for use in the compositions are various acid dyes such as black, blue, brown, green, orange, red, violet, and yellow. Examples of Acid Black are numbers 1 and 52. Suitable blues include Acid Blue 1, 3, 9, 62, and 74, including Lakes thereof. Examples of browns and greens include Acid Brown 13 and Acid Green 1, 25, and 50, respectively. Suitable oranges include Acid Orange 3, 6, 7, and 24. Suitable reds include Acid Red 14, 18, 27, 33, 35, 51, 52, 73, 87, 92, 95, 184, and 195. Suitable violets include Acid Violet 9 and 43. Suitable yellows include Acid Yellow 1, 3, 23, and 73. In each case the dyes may be Lakes thereof. Such Acid dyes are set forth on pages 13-23 of the CTFA Cosmetic Ingredient Handbook, Eighth Edition, 2000, which is hereby incorporated by reference in its entirety.

(iv). Direct and Disperse Dyes

Also suitable are various types of dyes referred to as direct dyes or disperse dyes. Suitable direct dyes include Direct Black 51, Direct Blue 86, Direct Red 23, 80, and 81; Direct Violet 48, and Direct Yellow 12. Such direct dyes are set forth on pages 469-471 of the CTFA Cosmetic Ingredient Handbook, Eighth Edition, 2000, which is incorporated by reference in its entirety.

Suitable disperse dyes include Disperse Black 9, Disperse Blue 1, 3, and 7; Disperse Brown 1, Disperse Orange 3, Disperse Red 11, 15, and 17; and Disperse Violet 1, 4, and 15. Such disperse dyes are as set forth on 491-493 of the CTFA Cosmetic Ingredient Handbook, Eighth Edition, 2000, which is hereby incorporated by reference in its entirety.

The semi-permanent dye composition may contain a variety of other ingredients such as surfactants, humectants, preservatives, pH adjusters, and so on.

(c ). Hair Bleach

The hair color may be a hair bleach, used for bleaching the entire head of hair or just sections of the hair as in highlighting. Such hair bleaches are usually formed by combining a persulfate bleach composition with a developer composition as described above.

(i) Persulfate Composition

A variety of persulfate compositions may be suitable, including those well known in the art. The persulfate composition may be in the powdered particulate or paste form and in general may have one or more of the ingredients set forth below. The persulfate composition preferably comprises a mixture of persulfate salts that are capable of bleaching the hair when combined with an oxidizing agent or developer, particulate fillers, and, if desired, inorganic particulate colorants. The persulfate composition used in the claimed method may be found in the powdered particulate form, or in the form of a cream or paste as described in U.S. Pat. No. 5,888,484; and U.S. patent application Ser. No. 09/774,890, filed Feb. 1, 2001, assigned to Revlon Consumer Products Corporation, naming Teresita Imperial as inventor, both of which are hereby incorporated by reference in their entirety. The typical ingredients found in the persulfate composition are further described herein.

(1). Persulfates

The persulfate composition comprises about 10-65%, preferably about 20-60%, more preferably about 25-55% by weight of the total bleach composition of one or more inorganic persulfates which may be alkali metal or alkaline earth metal persulfates, or ammonium persulfate. Preferably the persulfate composition comprises one or more of an alkali metal, alkaline earth metal, or ammonium persulfate. Examples of alkali metal persulfates include lithium, sodium, potassium, cesium, and the like. Examples of suitable alkaline earth metals include magnesium, calcium, and the like. Particularly preferred are sodium, potassium, and ammonium persulfates. The persulfates are generally in particulate form and have particle sizes ranging from about 0.1 to 200 microns. The persulfates should be reactive with the hydrogen peroxide or the other peroxide oxidizing agent present in the oxidizing agent composition when the persulfate composition is combined with the oxidizing agent composition.

(2). Alkalizing Agents

The persulfate composition preferably contains one or more alkalizing agents. Preferred alkalizing agents are one or more inorganic salts as set forth herein. Suggested ranges of inorganic salts are from about 0.1-40%, preferably about 0.5-35%, preferably about 1-30% by weight of the total composition.

(3). Particulate Fillers

The persulfate composition also preferably comprises one or more particulate fillers. Preferably, the bleach composition comprises about 5-60%, preferably about 8-55%, more preferably about 10-50% by weight of the total bleach composition of the particulate fillers. The term “particulate filler” means a generally inert particulate having a particle size of about 0.1-250 microns. The particulate fillers provide volume and, when mixed with the persulfates, dilute the persulfate particles. A variety of particulate fillers are suitable including inorganics, inorganic salts, hydrophilic colloids, carbohydrates, soaps, alkyl sulfates, and the like.

(i) Inorganics

Examples of inorganics include silica, hydrated silica, alumina, attapulgite, bentonite, calcium oxide, chalk, diamond powder, diatomaceous earth, fuller's earth, hectorite, kaolin, mica, magnesium oxide, magnesium peroxide, montmorillonite, pumice, talc, tin oxide, zeolite, zinc oxide, and the like.

(ii) Hydrophilic Colloids

Examples of suitable hydrophilic colloids include hydroxyethylcellulose, locust bean gum, maltodextrin, methylcellulose, agar, dextran, dextran sulfate, gelatin, pectin, potassium alginate, sodium carboxymethylchitin, xanthan gum, and the like.

(iii) Carbohydrates

Examples of suitable carbohydrates include sugars such as glucose, sucrose, maltose, xylose, trehelose, and derivatives thereof, in particular sugar esters of long chain, C₁₄₋₃₀ fatty acids, as well as dextrins, cellulosics, and derivatives thereof.

(iv) Soaps and Alkyl Sulfates

Examples of soaps and alkyl sulfate particles that may act as particulate fillers include the aluminum, sodium, and potassium salts of fatty acids such as aluminum distearate, aluminum isostearate, aluminum myristate, calcium behenate, calcium stearate, calcium behenate, magnesium stearate, magnesium tallowate, potassium palmitate, potassium stearate, potassium oleate, sodium stearate, sodium oleate, sodium myristate, sodium palmitate, and the like. Suitable alkyl sulfates include sodium lauryl sulfate, sodium cetyl sulfate, sodium myristyl sulfate, sodium octyl sulfate, and the like.

(v). Inorganic Colorants

If desired, the persulfate composition may comprise about 0.01-2%, preferably about 0.05-1%, more preferably about 0.1-1% by weight of the total persulfate composition of an inorganic colorant. The inorganic colorant is preferably in the particulate form and will provide a subtle coloration to the powder composition to make it more aesthetically pleasing for commercial purposes. Particularly preferred for use in the persulfate composition is ultramarine blue.

(dd). Lipophilic Ingredients

It may be desirable to include one or more lipophilic ingredients in the persulfate composition. If so, suggested ranges of the lipophilic ingredient are from about 0.1-70%, preferably about 0.5-60%, more preferably about 1-50% by weight of the total composition. Suitable lipophilic ingredients include liquids, solids, or semi-solids at room temperature (25° C.). Examples of such lipophilic ingredients include silicone oils, organic oils, fatty acids, fatty alcohols, hydrocarbons, paraffins, silicone waxes, and the like.

2. The After Treatment Compositions

Optionally, the hair that is pretreated and colored in the method of the invention may be treated with compositions such as shampoo or conditioner or both after completion of the coloring step. After the colorant is removed from the hair by rinsing, the hair may be shampooed or conditioned or both.

(a). After Treatment Shampoo

Any after treatment shampoo is generally suitable provided it does not remove the color from the hair. Suitable shampoo compositions are generally comprised of from about 1-99%, preferably from about 5-95%, more preferably from about 10-90% by weight of the total composition of water, and from about 0.1-99%, preferably from about 1-95%, more preferably from about 5-90% by weight of the total composition of a cleansing surfactant. Suitable cleansing surfactants are generally anionic, amphoteric, betaine, or zwitterionic surfactants. Preferably, anionic surfactants include alkyl ether or alkyl ether sulfates such as sodium laureth-sulfate, sodium lauryl sulfate, and the like.

(b). After Treatment Conditioner

Any after treatment conditioner is generally suitable as well, provided it conditions the hair and does not have any adverse effect on the color. Such conditioners generally contain from about 0.1-99%, preferably from about 0.5-95%, more preferably from about 1-90% by weight of the total composition of water and from about 0.1-99%, preferably from about 0.5-95%, more preferably from about 1-90% by weight of the total composition of cationic conditioning agents, e.g. cationic surfactants or cationic polymers. The conditioner may contain fatty acids or alcohols, silicone oils, and the like. Suitable hair conditioners are as set forth in U.S. Pat. No. 5,989,533, which is hereby incorporated by reference in its entirety.

In the method of the invention the leave on pretreatment composition is applied to the hair liberally, working it through the hair strands by combing, for example, ensuring that the ends of the hair are coated. The desired coloring is conducted after application of the pretreatment composition and without washing it out of the hair. The pretreatment composition may be applied to wet or dry hair, but preferably it is applied to dry hair.

When hair is treated using the method and kits of the invention the resulting hair exhibits improved conditioning. Also, the color deposition on the colored hair tends to be more even. Without being bound by explanation, it is believed that the pretreatment composition preferentially coats the hair depending on the degree of porosity and damage. When the hair is colored, the color itself is prevented from overdepositing on the porous sections of the hair.

The invention will be further described in connection with the following examples which are set forth for the purposes of illustration only.

### EXAMPLE 1

Pretreatment compositions were made as follows:

% by weight

| Ingredient | 1 | 2 | 3 | 4 |
|---|---|---|---|---|
| Cocos Nucifera (coconut) oil | 100 | 50 | 50 | 50 |
| Mineral oil | — | 50 | — | — |
| Isopropyl palmitate | — | — | 50 | — |
| Isopropyl myristate | — | — | — | 50 |

### EXAMPLE 2

Five salon panelists were recruited for this study, the panelists were tested using the pretreatment composition of the invention with standard oxidative hair color. The tests were conducted split head on panelists that short, medium, or long hair.

The half head of each panelist was treated with the pretreatment composition directly followed by an oxidative hair color procedure without removing the pretreatment composition from the hair. The oxidative dye was selected to most closely match the natural hair color of each panelist. Two of the panelists were treated with Revlon High Dimension Hair Color No. 54, Bronzed Brown. One panelist was treated with Revlon Colorsilk Medium Golden Chestnut Brown, 4GM. Two of the panelists were treated with Revlon Colorsilk Dark Auburn, 3R.

The ingredient listing from each of the products, as set forth on the product boxes, is set forth below:

Revlon High Dimension Hair Color No. 54, Bronzed Brown, SKU No. 6240-54

Color Developer: Water, Hydrogen Peroxide, Cetearyl Alcohol, Ceteareth-20, Mineral Oil, Cyclomethicone, Trimethylsiloxysilicate, Trimethylsilylameodimethicone, C11-15 Pareth-7, C12-16 Pareth-9, Trideceth-12, Glycerin, Steareth-10 Allyl Ether/Acrylates Copolymer, Lauramide MEA, Disodium Phosphate, Phosphoric Acid, EDTA, Methylparaben. 12825
Vitalizing Conditioner: Water, Cetearyl Alcohol, Glycerin, Behentrimonium Chloride, Amodimethicone, Cetrimonium Chloride, Trideceth-12, Panthenol, Sodium Benzotriazolyl Butylphenol Sulfonate, Buteth-3, Tributyl Citrate, Cholesteryl Oleyl Carbonate, Cholesteryl Nonanoate, Cholesteryl Chloride, Isostearyl Citrate/Glycolate/Lactate/Malate, Polyethylene Terephthalate, Acrylates Copolymer, Citric Acid, Mangifera Indica Seed Butter (Mango), Fragrance, Methylparaben, Propylparaben, Methylchloroisothiazolinone, Methylisothiazolinone. 12824
Creme-Gel Color: Water, MEA-Oleate, Ethoxydiglycol, Cetearyl Alcohol, Ammonium Hydroxide, Emulsifying Wax, Fragrance, Oleth-20, Steareth-21, Lmnanthes Alba (Meadowfoam) Seed Oil, Oleyl Alcohol, Polyquaternium-10, Polyquaternium-28, Hydrolyzed Wheat Protein, Sodium Benzotriaolyl Butylphenol Sulfonate, P-Phenylenediamine, P-Aminophenol, 4-Amino-2-Hydroxytoluene, Resorcinol, Ammonium Lauryl Sulfate, Erythorbic Acid, Hypnea Musciformis Extract, Gellidiela Acerosa Extract, Sargassum Filipendula Extract, Sorbitol, Buteth-3, Tributyl Citrate, Ethanolamine, Sodium Sulfite, Tetrasodium EDTA, Mica, Titanium Dioxide.
   Revlon Colorsilk Medium Gold Chestnut Brown, 4GM. SKU No. 6623-46
COLORANT: Water; MEA-Oleate; Isopropyl Alcohol; Ethanolamine; PEG-2 Cocamine; Lauramide MEA; Sodium Laureth Sulfate; Potassium Cocoyl Hydrolyzed Collagen; Wheat Amino Acids; Hypnea Musciformis Extract; Gellidiela Acerosa Extract; Sargassum Filipendula Extract; Sorbitol; Oleyl Alcohol; Meadowfoam (Limnanthes Alba) Seed Oil; Polyquaternium-28; Laneth-5; Sodium Benzotriazolyl Butylphenol Sulfonate; Buteth-3; Tributyl Citrate; Sodium Sulfite; Erythorbic Acid; Tetrasodium EDTA; Fragrance; p-Phenylenediamine; 4-Amino-2-Hydroxytoluene; Resorcinol; 2-Methylresorcinol; Phenyl Methyl Pyrazolone; p-Aminophenol; m-Aminophenol.
CREAM DEVELOPER: Water; Hydrogen Peroxide; Cetearyl Alcohol; Lauryl Pyrrolidone; Steareth-10 Allyl Ether/Acrylate Copolymer; Trimethylsilylamodimethicone; Ceteareth-20; Cyclomethicone; Trimethylsiloxysilicate; C11-15 Pareth-7; C12-16 Pareth-9; Trideceth-12; Glycerin; Mineral Oil; EDTA; Methylparaben; Disodium Phosphate; Phosphoric Acid.
AFTER COLOR CONDITIONER: Water; Behentrimonium Chloride; Cetearyl Alcohol; Linoleamidopropyl Dimethylamine Dimer Dilinoleate; Stearyl Alcohol; Methoxypropylgluconamide; Panthenol; Panthenyl Ethyl Ether; Dimethicone Copolyol Meadowfoamate; Amodimethicone; Trideceth-12; Isostearyl Citrate/Glycolate/Lactate/Malate; Diisostearoyl Trimethylolpropane Siloxy Silicate; Hydroxyethylcellulose; Cocamidopropyl Betaine; Ceteareth-20; Cetyl Alcohol; Stearamidopropyl Dimethylamine; Cetrimonium Chloride; Citric Acid; Methylparaben; Propylparaben; Methylchloroisothiazolinone; Methylisothiazolinone; Fragrance
   Revlon Colorsilk Dark Auburn, 3R, SKU No. 6623-31
COLORANT: Water; MEA-Oleate; Isopropyl Alcohol; Ethanolamine; PEG-2 Cocamine; Lauramide MEA; Sodium Laureth Sulfate; Potassium Cocoyl Hydrolyzed Collagen; Wheat Amino Acids; Hypnea Musciformis Extract; Gellidiela Acerosa Extract; Sargassum Filipendula Extract; Sorbitol; Oleyl Alcohol; Meadowfoam (Limnanthes Alba) Seed Oil; Polyquaternium-28; Laneth-5; Sodium Benzotriazolyl Butylphenol Sulfonate; Buteth-3; Tributyl Citrate; Sodium Sulfite; Erythorbic Acid; Tetrasodium EDTA; Fragrance; p-Phenylenediamine; 2-Methylresorcinol; Resorcinol; p-Aminophenol; o-Aminophenol; 4-Amino-2-Hydroxytoluene.
CREAM DEVELOPER: Water; Hydrogen Peroxide; Cetearyl Alcohol; Lauryl Pyrrolidone; Steareth-10 Allyl Ether/Acrylate Copolymer; Trimethylsilylamodimethicone; Ceteareth-20; Cyclomethicone; Trimethylsiloxysilicate; C11-C15 Pareth-7; C12-16 Pareth-9; Trideceth-12; Glycerin; Mineral Oil; EDTA; Methylparaben; Disodium Phosphate; Phosphoric Acid. AFTER
COLOR CONDITIONER: Water; Behentrimonium Chloride; Cetearyl Alcohol; Linoleamidopropyl Dimethylamine Dimer Dilinoleate; Stearyl Alcohol; Methoxypropylgluconamide; Panthenol; Panthenyl Ethyl Ether; Dimethicone Copolyol Meadowfoamate; Amodimethicone; Trideceth-12; Isostearyl Citrate/Glycolate/Lactate/Malate; Diisostearoyl Trimethylolpropane Siloxy Silicate; Hydroxyethylcellulose; Cocamidopropyl Betaine; Ceteareth-20; Cetyl Alcohol; Stearamidopropyl Dimethylamine; Cetrimonium Chloride; Citric Acid; Methylparaben; Propylparaben; Methylchloroisothiazolinone; Methylisothiazolinone; Fragrance

The pretreatment composition of Formula 1, Example 1 was applied to half heads on each panelist. The composition was liberally applied to the select hair strands by combing through to ensure even distribution, particularly on hair ends, and avoiding the root area. Leaving the pretreatment composition on the hair, the entire head of hair was oxidatively colored according to kit instructions. After the oxidative dye process was completed, trained evaluators rated attributes such as color, lightening effects, conditioning, and so on, noting the differences between the untreated half head and the half head treated with the pretreatment composition.

In general, the half head treated with the pretreatment composition exhibited improved wet hair properties such as smoothness and slip, and initial and repeated comb through. The dry half head treated with the pretreatment composition was generally easier to blow dry and style, had improved slip and comb through, luster, and style control and manageability. The panelists preferred the properties of the half head treated with the pretreatment composition. After three months, the panelists' hair was reevaluated. In one panelist the oxidative hair color retention on the half head treated with the pretreatment composition was better than that found on the untreated half head. In addition, for all panelists, the half head pretreated with the pretreatment composition appeared healthier and exhibited improved evenness of color from root to tip. Accordingly, it appeared that the pre-treatment prevented over deposition of color on the ends of the hair. Also, there was no perceptible difference in color deposit on the hair roots (new hair growth) between untreated heads and heads treated with the pre-treatment composition. This indicates that the pre-treatment composition is absorbed into the hair where it is needed, such as porous areas.

### EXAMPLE 3

Four salon panelists were recruited for this study. The panelists were tested using Revlon Frost & Glow Bleach Kit, SKU No. 1565-06 (off scalp bleach) and the pretreatment composition of Formula 1, above. The tests were conducted split head on panelists having short, medium, and long hair.

The kit used to bleach the hair contained the following:

Frosting Powder

Cream Developer

After Highlighting Shampoo

After Highlighting Conditioner

LightZones Cap

Over Cap

Applicator Spatula

Special Hair strand Hook

Mixing Bowl

Protective Gloves

Instructions

The ingredient listing for the compositions in the kit, as set forth on the product box, are reproduced below:
Blonding Powder Contains: Potassium Persulfate, Sodium Silicate. Sodium Metasilicate, Sodium Persulfate, Sodium Stearate, Hydroxyethylcellulose, EDTA, Hydrated Silica. 12821 Blonding Cream Developer Contains: Water, Hydrogen Peroxide, Cetearyl Alcohol, Laureth 23, Sodium Lauryl Sulfate, Sodium Cetearyl Sulfate, Disodium Phosphate, Phosphoric Acid, EDTA.
Cream Developer Contains: Water, Hydrogen Peroxide, Cetearyl Alcohol, Mineral Oil, Cyclomethicone, Trimethysiloxysilicate, Trimethylsilamodimethicone, C11-15 Pareth-7, C12-16 Pareth-9, Trideceth-12, Glycerin, Ceteareth-20, Steareth-10 Allylether/Acrylate Copolymer, Disodium Phosphate, Phosphoric Acid, EDTA, Methylparaben. 11336
Blonding Oil Contains: Water, Oleic Acid, Laureth-4, Isopropyl Myristate, Isopropyl Alcohol, Ethoxydiglycol, Ethanolamine, Oleylalcohol, Simmondsia Chinsensis (Jojoba) Seed Oil, Fragrance, Tetrasodium EDTA.
After-Highlighting Shampoo Contains: Water, Ammonium Lauryl Sulfate, Sodium Laureth Sulfate, Lauramide MEA, Methoxypropylgluconamide, Hydrolyzed Silk, Panthenol, Tocopheryl Acetate, Retinyl Palmitate, Peg-12 Dimethicone, Cocamidopropyl Betaine, Sodium C14-17 Alkyl Sec Sulfonate, Stearamide Oxide, Hydroxypropyl Methycellulose, PEG-23M, Propylene Glycol, Sodium Chloride, Citric Acid, Tetrasodium EDTA, Methylparaben, Propylparaben, Methylchloroisothiazolinone, Methylisothiazolinone, Fragrance, FD&C Yellow #5, FD&C Red #4, D&C Red #33, FD&C Yellow #6. 20029
After-Blonding Conditioner Contains: Water, Behentrimonium Chloride, Ceteryl Alcohol, Linoleamidopropyl Dimethylamine Dimer Dilinoleate, Stearyl Alcohol, Cetyl Alcohol, Panthenol, Panthrnyl Ethyl Ether, Cocamidopropyl Betaine, Stearamidopropyl Dimethylamine, Amodimethicone, Diisostearoyl Trimethylolpropane Siloxy Silicate, Isostearyl Glycolate, Triisostearyl Citrate, Diisostearyl Malate, Isostearyl Lactate, Dimethicone PEG-8, Meadowfoamate, Methoxypropylgluconamide, Ceteareth-20, Hydroxyethylcellulose, Trideceth-12, Cetrimonium Chloride, Citric Acid, Methylparaben, Propylparaben, Methychloroisothiazolinone, Methylisothiazolinone, Fragrance.

The lightzones cap was placed over the hair. Hair was pulled through the holes in the cap using the special hair strand hook. The pretreatment oil, Formula 1, from Example 1, was applied to hairs pulled through the holes in the lightzones cap on half of the head of each panelist. The half head was left untreated with the pretreatment oil. The pretreatment oil was liberally applied to the strands and worked through the hair strands and ends to ensure even coverage.

Then the hair was bleached in the usual manner according to the instructions in the kit, without rinsing the pretreatment composition from the hair. After completing the shampoo and conditioner steps, the lightzone cap was removed from the head of each panelist and trained evaluators rated attributes such as color, lightening effects, conditioning, and so on.

In general, the lightzone cap was more easily removed from the half heads that were treated with the pretreatment composition and the pre-treated hair was easier to blow dry.

Three months after the procedure, the panelists were evaluated again. The hair strands on the half head treated with the pretreatment composition were in significantly better condition that the untreated hair strands.

### EXAMPLE 4

Four salon panelists were recruited for this study. The panelists were treated with on scalp hair bleach, Revlon Frost & Glow Blonding Kit, SKU no. 7562-01. The tests were conducted split head on panelists having short, medium, and long hair.

The items present in the kit:

Applicator Tip

Blonding Powder

Blonding Cream Developer

Blonding Oil

After-Blonding Conditioner

Protective Gloves

Instructions

The ingredient list for the compositions contained in the kit as set forth on the box, are reproduced below.
Blonding Powder Contains: Potassium Persulfate, Sodium Silicate. Sodium Metasilicate, Sodium Persulfate, Sodium Stearate, Hydroxyethylcellulose, EDTA, Hydrated Silica. 12821 Blonding Cream Developer Contains: Water, Hydrogen Peroxide, Cetearyl Alcohol, Laureth 23, Sodium Lauryl Sulfate, Sodium Cetearyl Sulfate, Disodium Phosphate, Phosphoric Acid, EDTA.
Blonding Oil Contains: Water, Oleic Acid, Laureth-4, Isopropyl Myristate, Isopropyl Alcohol, Ethoxydiglycol, Ethanolamine, Oleylalcohol, Simmondsia Chinsensis (Jojoba) Seed Oil, Fragrance, Tetrasodium EDTA.
After-Blonding Conditioner Contains: Water, Behentrimonium Chloride, Ceteryl Alcohol, Linoleamidopropyl Dimethylamine Dimer Dilinoleate, Stearyl Alcohol, Cetyl Alcohol, Panthenol, Panthrnyl Ethyl Ether, Cocamidopropyl Betaine, Stearamidopropyl Dimethylamine, Amodimethicone, Diisostearoyl Trimethylolpropane Siloxy Silicate, Isostearyl Glycolate, Triisostearyl Citrate, Diisostearyl Malate, Isostearyl Lactate, Dimethicone PEG-8, Meadowfoamate, Methoxypropylgluconamide, Ceteareth-20, Hydroxyethylcellulose, Trideceth-12, Cetrimonium Chloride, Citric Acid, Methylparaben, Propylparaben, Methychloroisothiazolinone, Methylisothiazolinone, Fragrance.

The pretreatment composition Formula 1 of Example 1 was applied to half heads on each panelist. The pretreatment composition was liberally applied and combed through to ensure even distribution. The bleach product was applied to each panelist according to the instructions in the kit without rinsing the pretreatment composition from the hair. After completion of the bleach product process, the hair was dried, styled, and evaluated. Trained evaluators rated attributes such as color, lightening effects, conditioning, and so on.

It was noted that the half head treated with the pretreatment composition was easier to style and blow drying was easier. The bleached hair treated with the pretreatment composition exhibited more even color throughout the strands including the ends.

After three months the panelists were evaluated again. The hair treated with the pretreatment composition exhibited improved quality and condition when compared to strands on the untreated side.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. An oxidative hair color kit comprising at least: (a) a receptacle containing an oxidative hair color composition, (b) a receptacle containing a developer composition, wherein (a) and (b) are combined to form a hair color mixture; and (c) a receptacle containing at least one leave on pretreatment composition for application to the hair prior to coloring the hair with the mixture of (a) and (b).

2. The kit of claim 1 wherein the leave on pretreatment composition comprises a low molecular weight diffusible oil.

3. The kit of claim 2 wherein the low molecular weight diffusible oil comprises coconut oil, mineral oil, esters having a molecular mass of 200 to 600 grams/mol, or mixtures thereof.

4. The invention is further directed to a hair bleach kit comprising (a) a receptacle containing a developer, (b) a receptacle containing a persulfate bleach composition, and (c) a receptacle containing a leave on pretreatment composition for application to the hair prior to bleaching the hair with the hair bleach mixture formed by combining (a) and (b).

5. The kit of claim 4 wherein the bleach is in a powdered form.

6. The kit of claim 4 wherein the pretreatment composition comprises a low molecular weight diffusible oil.

7. The kit of claim 6 wherein the oil is coconut oil, mineral oil, or esters having a molecular mass of 200 to 600 grams/mol, or mixtures thereof.

8. A semi-permanent hair color kit comprising (a) a receptacle containing a semi-permanent dye composition, and (b) a receptacle containing a leave on pretreatment composition for application to the hair prior to coloring the hair with the semi-permanent dye composition.

9. The kit of claim 8 wherein the leave on pretreatment composition comprises a low molecular weight diffusible oil.

10. The kit of claim 9 wherein the oil comprises coconut oil, mineral oil, esters having a molecular mass of 200 to 600 grams/mol, or mixtures thereof.

11. A hair highlighting kit comprising (a) a receptacle containing a developer composition, (b) a receptacle containing a persulfate bleach composition, (c) at least one application device, and (d) a receptacle containing a leave on pretreatment composition for application to the hair prior to highlighting the hair with the mixture of (a) and (b).

12. The kit of claim 11 wherein the pretreatment composition comprises coconut oil, mineral oil, or esters having a molecular mass of 200 to 600 grams/mol.

13. A lowlighting kit comprising (a) a receptacle containing a developer composition, (b) a receptacle containing a lowlight dye composition, (c) an applicator; and (d) a receptacle containing a leave on pretreatment composition for application to the hair prior to applying lowlights to the hair with the applicator and the mixture of (a) and (b).

14. The kit of claim 13 wherein the pretreatment composition comprises coconut oil, mineral oil, or esters having a molecular mass of 200 to 600 grams/mol.

15. A method for permanently coloring hair comprising:
(a) applying a leave on pretreatment composition to the hair to be colored,
(b) coloring the pretreated hair with a hair color composition.

16. The method of claim 15 wherein the pretreatment composition comprises a low molecular weight diffusible oil.

17. The method of claim 16 wherein the oil comprises coconut oil, mineral oil, or mixtures thereof.

18. The method of claim 15 wherein the hair coloring composition is an oxidative hair color mixture prepared by combining a developer and oxidative hair color composition.

19. The method of claim 15 wherein the hair coloring composition is a hair bleach mixture prepared by combining a powdered bleach composition and a developer.

20. A method for improving hair conditioning and/or evenness of color deposition in hair that is permanently colored comprising applying a leave on pretreatment composition containing a low molecular weight diffusible oil to the hair prior to coloring the hair.

21. The method of claim 20 wherein the permanent coloration is an oxidative dye or bleach.

22. The method of claim 20 wherein the pretreatment composition comprises a low molecular weight diffusible oil.

23. The method of claim 22 wherein the oil comprises coconut oil, mineral oil, or mixtures thereof.
